(19) United States
(12) Patent Application Publication
(10) Pub. No.: US 2006/0172376 A1
(43) Pub. Date: Aug. 3, 2006

(54) Method and device for the continuous production of biomolecules

(76) Inventors: Hassan Chadjaa, Madeleine (CA); Mohamed Rahni, Shawinigan, QC (US)

Correspondence Address: OGILVY RENAULT LLP, 1981 MCGILL COLLEGE AVENUE, SUITE 1600, MONTREAL, QC, H3A2Y3, CA

(21) Appl. No.: 10/545,272

(22) PCT Filed: Feb. 11, 2004
(86) PCT No.: PCT/CA04/00184

§ 371 Date: Feb. 22, 2006

Related U.S. Application Data
(60) Provisional application No. 60/446,274, filed on Feb. 11, 2003.

(51) Int. Cl. C12P 21/06; C12N 1/21; C07K 14/195

(52) U.S. Cl. 435/69.1; 435/252.3; 435/291.1; 530/350

(57) ABSTRACT

The invention relates to a method and device for the continuous production of endogenous or recombinant native polypeptides. The method and device comprise the steps of cultivating recombinant microorganisms in a bioreactor, withdraw a sample of the microbial suspension in the reactor in a continuous manner and subject the same to a first filtration to separate the biomolecules from the microbial suspension and to a second filtration to separate the biomolecules of interest form the other biomolecules.

### TECHNICAL FIELD

The present invention relates to a process and a device that are used for the production of recombinant bio-molecules. In particular, this process and this device allow for the continuous production of bio-molecules, by using growth micro-organisms in a bioreactor, and means for the continuous sampling of a specific quantity of a suspension of micro-organisms to subject same, in a continuous manner, to filtration steps making it possible to obtain purified or concentrated bio-molecules.

### PRIOR ART

Biotechnologies represent a strategic research axis of the utmost importance. The large companies involved in biotechnology, that have been recorded, produce recombinant bioreactors for the production of recombinant or natural bio-molecules of high added values. Many companies concentrate their efforts on the production in bulk of biological molecules (essentially protean), of pharmaceutical, cosmetic or medical interest. There are numerous examples of these molecules: monoclonal antibodies, HLA, P53 molecules and interferons (anti-carcinogenic), insulin, antibiotics, etc. The list of molecules grows as the days go by and their applications are increasingly wide.

Most companies involved in biotechnology concentrate their research efforts in the production of transgenic bioreactors or those issued from selection processes, such as bacteria, yeasts, fungi, plants and animals. However, research efforts are much more restricted in the field of extraction and purification of natural or recombinant molecules produced.

Extraction and purification of molecules represent the main restraints to the development of the majority of production technologies. As a matter of fact, the companies that include in their research objectives the production, extraction and purification of recombinant molecules, in addition to technical difficulties, face extremely high costs associated with the processes of extraction and of chemical purification.

In order to overcome these disadvantages, some researchers have developed production models according to other approaches, such as transgenic animals and plants. As compared to cellular bioreactors, the models which have been developed present disadvantages that are more and more important. As a matter of fact, managing these systems causes problems because of environmental, public health, image constraints, in addition to being extremely costly. The result is that, even if most of the companies have developed processes for the production of bio-molecules on a laboratory scale, they do not prompt their research to a semi-industrial or industrial pilot scale since the constraints associated with production cost imply that these researches do not exceed laboratory phase. The primary objective that is retained in most cases is to ensure the production of the target molecule on a cellular scale, and to validate same.

The bioreactor with external membranes (BRM) has proven itself in applications such as sewage treatment. However, its use in biotechnological applications is practically non-existent to this day. The first membrane bioreactors combined purification capacities for micro-organisms and the separation possibilities of membranous filtration techniques. It is based on the use of a membrane type of bioreactor in which the secondary decantation is replaced by a tangential micro-filtration. Filtration allows the particles to be maintained in suspension according to their size. The process is characterized by being compact and modular.

Batch production has many disadvantages as compared to a production in continuous mode. The most important consists in operation costs that are higher as compared to a production in continuous mode, a lower production yield, longer maintenance and preparation (monitoring) and consequently shorter times of use. The latter point represents more important capitalizations. Finally, the continuous mode allows a greater production capacity than the batch mode. In the case of this platform, the optimized production capacity in continuous mode is five (5) times higher than that of a production in batch mode.

It would therefore be advantageous to be able to rely on a method of extraction and purification of recombinant or native bio-molecules that would make it possible to reduce the cost inherent to this step of production of such molecules. In addition, it would be advantageous to be able to rely on a method that permits the continuous production of bio-molecules of different molecular weights, by cultivating micro-organisms (bacteria, yeasts, mildews) or plant cells and animal cells, expressing genes of interest that originate from different sources (plants, animals, human, etc).

### SUMMARY OF THE INVENTION

A first object of the present invention consists in a process for the continuous production of bio-molecules of interest with micro-organisms. Generally, this process comprises the following steps:
a) cultivating micro-organisms expressing the recombinant bio-molecules of interest in a bioreactor;
b) transferring a portion of the suspension of producing micro-organisms and a portion of the medium in a container;
c) separating and concentrating the microbial biomass of the culture medium issued from bio-fermentation; in the case of exogenous molecules, the recombinant bio-molecules that are produced are present in the filtration ultra-filtrate; in the case of endogenous molecules, the bio-molecules are released through a cellular lysis (sonication);
d) in the case of secreted bio-molecules (exogenous), the sequence(s) of membranous filtration consist(s) in separating, concentrating and purifying the recombinant bio-molecules of interest from the substances and other nutritive elements that constitute the medium (amino-acids, organic acids, mineral elements, etc); in the case of molecules that are not secreted (endogenous), the bio-molecules of interest are released from the cellular compartments through a lysis (sonication) and the membranous filtration sequences consist, in a first step, in separating the bio-molecules of interest from the cellular lysate (cellular debris, constituent proteins, etc) and in a second step, in separating, concentrating and purifying the bio-molecules of interest from the remaining medium (ultra-filtrate from the first filtration step); and
e) collecting the bio-molecules of interest.

The invention also concerns a device for the continuous production of microbial bio-molecules comprising:
a) a bioreactor;
b) a source of nutrients, and means for introducing said nutrients in the bioreactor;
c) means for aerating the bioreactor;
d) means for agitating the bioreactor;
e) a first transient vat and means for introducing the suspension of producing micro-organisms in said transient vat;
f) a first filtration means and means for introducing the suspension of micro-organisms in the first filtration means from the first transient vat;
g) a second container for suspending bio-molecules therein and a means for introducing the suspension of bio-molecules in the container after a first filtration;
h) a second filtration means and means for, introducing the suspension of bio-molecules having undergone a first filtration in the second filtration means, from the second container; and
i) a third container for suspending bio-molecules of interest therein and means for introducing the solution of bio-molecules of interest in the container after the second filtration.

Another object of the present invention consists in a process for the continuous production and separation of target polypeptides by means of a micro-organism comprising the steps of:
a) causing a micro-organism producing at least one target polypeptide to grow in a bioreactor autonomously supplied with a culture medium for a predetermined period of time;
b) allowing a portion of the culture medium to be autonomously transferred in a first container, so that this container promotes the accumulation of volumes extracted from the bioreactor in compensation for the volumes of fresh medium added in a continuous manner, the container being refrigerated at 4° C. to allow for the accumulation and the residence of a mixture of cells and culture broth before their sonication, in a second refrigerated container of same capacity and kind as the first container, that is used to preserve the mixture at 4° C. after sonication;
c) self-inducing separation of the target polypeptides from the portion of culture medium by at least one passage in at least one membrane, selected (nature and cutting threshold) according to the filtration objectives, the characteristics of the solution to be filtered and the operation criteria, the membrane preferably being selected from micro-filtration or ultra-filtration ceramic membranes;
d) separating the target polypeptides from the microbial molecules by means of a passage through a second membrane; and
e) collecting the target polypeptides.

Said bioreactor may be a bioreactor with external membrane.

The target polypeptides are preferably native or recombinant proteins and are produced in the culture medium or in endogenous manner, i.e. they are accumulated, in the producing cell. They may be selected from enzymes, transport proteins, anti-oxidizing proteins or food proteins.

The micro-organisms that can be used to carry out the present invention may be selected from the group of bacteria, yeasts, mildews, virus, a protozoan, a fungus, or yeast, a plant cell, an animal cell, or an insect cell.

Growth of the producer cells preferably corresponds, but not exclusively, to a point at the end of the logarithmic growth phase, with an optic density (DO) of 1.8, at a concentration of 0.2×10¹⁰ cells per milliliter, this cellular concentration being obtained between 7 and 8 hours after the start of bio-fermentation and 2 hours after the start of the pumps provided for the transfers between the bioreactor and the first and second containers, the start of the operation of the pumps provided for adding fresh medium being carried out at a flow speed of 37 ml/min, for the type of fermenter used, and optimized for purging at an equivalent flow, and the average time for initiating the operation of the pumps being determined to be at 5 hours and 30 minutes after the start of the fermentation, the DO being equal to 1.5, the DO and the cell concentration remaining stable and constant (variability <10% calculated on a base of 30 fermentations) during the entire step of production in the bio-fermenter.

A device for the continuous production of at least one target polypeptide comprising:

a bioreactor;

a source of culture medium and nutrient substrates, and means permitting to introduce them in the bioreactor;

means for ventilating the bioreactor;

means for stirring the culture medium in the bioreactor;

a first container adapted for sterile preservation of the culture medium and means allowing its autonomous and continuous transfer in said bioreactor;

at least one first filtration means (micro-filtration) and means for separating the cells (biomass) in the concentrate from the partially consumed culture medium (ultra-filtrate) through a filtration means (micro-filtration from a bio-fermenter; means operative so that after membranous separation, the concentrate remains in the first container and the ultra-filtrate is sent towards a second container, this second container being arranged to produce a suspension of filtered target polypeptide;

means for introducing the suspension of filtered target polypeptide in the second container after a first filtration, the second container allowing recovery and residence of the ultra-filtrate under sterile conditions at a temperature of 4° C., this ultra-filtrate constituting the portion that contains the target protein when it is released in the culture medium or in the cellular compartment, the latter being released after lysis by sonication, the separation being carried out after the sonication step, the target polypeptide finding itself in the ultra-filtrate after separation by micro-filtration;

at least one second filtration means (ultra-filtration ceramic membrane) and means for introducing the polypeptide suspension having undergone a first filtration in the second filtration means from the second container; and

means for collecting target polypeptide having undergone at least one second filtration.

Said bioreactor is preferably, but not exclusively, a bioreactor with external membrane, the first filtration means being a ceramic membrane, the second filtration means also being a membrane, which itself may alternately or cumulatively or in combination be a micro-filtration, ultra-filtration, nano-filtration, reverse osmosis or dialysis membrane.

### BRIEF DESCRIPTION OF THE FIGURES

FIG. 1 shows a diagram illustrating a device for the production of recombinant molecules that can be used for carrying out the present invention.

FIG. 2 illustrates a device for the production of recombinant molecules expressed in the intracellular compartments of bacteria;

FIG. 3 illustrates a diagram of different filtration steps allowing the separation, concentration and purification of a molecule produced in endogenous manner in a bacterium;

FIG. 4 illustrates an example of diagram of different steps of filtration allowing the separation, concentration and purification of a secreted molecule;

FIG. 5 illustrates the evolution of the flow of the ultra-filtrate as a function of the rate of concentration during the separation of the cellular debris;

FIG. 6 illustrates the evolution of the flow of the ultra-filtrate as a function of the rate of concentration during GFP separation; and

FIG. 7 illustrates the evolution of the total proteins and of the COT in the concentrate during diafiltration.

### DESCRIPTION OF PREFERRED EMBODIMENTS

In accordance with the present invention, a technological platform for the production, extraction and purification of protein bio-molecules in continuous mode is developed. The process and the device are used for the production and the purification of molecules secreted by microbial cells and molecules that are expressed in different cellular compartments. For this last category of molecules, the extraction and purification may require an additional step of lysis of the micro-organism, independently of the compartment where the molecule is found in the cell.

The technology used is based on the production of micro-organisms that produce the bio-molecules of interest and on the extraction/purification of these bio-molecules by filtration on membranes. These two technologies are associated to develop a technological platform of production and extraction/purification.

The production/separation system of the present invention is preferably made of a process and technology sequences making it possible to prepare, extract and purify, continuously or in uninterrupted manner, the protein molecules of interest by means of a bioreactor associated with membranous technologies.

The membranous technologies that include micro-filtration, ultra-filtration, nano-filtration and reverse osmosis, once the molecule has been produced and is secreted in the medium, operate to separate, extract and purify the molecule according to required purity parameters. One skilled in the art will understand that these methods may also be used when the micro-organisms were previously treated with lysis.

The system may be used for the continuous production of exogenous and/or endogenous bio-molecules for various applications, namely as bio-pesticides (bactericides, fungicides, etc), nutraceutical agents and functional foods (probiotic), agri-food (additives, food supplements, enzymes, digestion aids, etc) and pharmaceutical agents.

Contrary to membrane bioreactors used in the treatment of sewage, the present invention does not provide for the return of bacteria in the fermenter (bioreactor). Moreover, the claimed production system is in continuous mode, while the systems used in the treatment of sewage are stopped when the sludge comes to maturity, i.e. when the biological treatment comes to a close. In this case, reference is made to fermentation in discontinuous mode. Moreover, the microbial strains that are found in the treatment of sewage are heterogeneous while the biological systems used for the growth and continuous production of bio-molecules are pure strains and, in most cases, are preferably recombinant. Continuous growth and production is made possible by coupling the bioreactor with the membranes, the latter being arranged either in series, or in parallel, depending on needs (system with variable geometry). The nutrients are continuously added in the bioreactor and an equivalent volume of culture broth is sampled in a synchronous manner to be sent towards a system of filtration with membranes.

The nature of the molecules of interest that can be produced by the method and system of the present invention are essentially protein molecules. The latter may be food proteins or those provided with a biological activity such as enzymes (proteases, lipases, oxydase, etc) or transport proteins (hemoglobin, myoglobin, transferin) or antioxidants (cosmetics). recombinant proteins that originate from the agri-food sector offer very large possibilities.

With respect to the production of protein bio-molecules, a unicellular fermenter is optimized for a continuous production of protein molecules. Extraction is carried out by means of a membranous system in order to separate the molecules from the production cells. Determination of the factors allowing the continuous operation of the bioreactor, its control and its optimization, are based on the following parameters: pH, temperature, oxygen diffusion, dilution volumes, etc. The phase that follows, while taking into account the choice of membrane previously done, allows one to extract, concentrate and purify the obtained bio-molecule.

The biological molecules that the system can produce are molecules secreted in the medium and the non secreted molecules. Separation, concentration and purification of the secreted bio-molecules do not require a step of continuous cellular lysis, while such a step is required for the treatment of non secreted molecules.

The main aspects to be controlled and optimized in order to maximize the yield of the operations are the following:

The nutrients should be appropriate and in adequate quantity to maximize the production of bio-molecules;

The bioreactor should contain the micro-organisms that produce the bio-molecules to be extracted and purified;

Ventilation should be optimized as a function of the respiratory mode and ratio of the micro-organisms;

The reactor should be provided with a system that is preferably but not exclusively of cylindrical geometrical shape, that has been optimized as a function of the production system to be used in order to provide optimal stirring and homogenization of the medium and a good diffusion of oxygen therein;

The pressure pump may be of various kinds, however a centrifugal type of pump gives better results;

The external membranes are selected on the basis of the bio-molecule that is intended to be extracted.

The first membranous sequence and this, independently of the type of molecule produced, is a membrane selected with a cutting threshold that allows the bio-molecule to pass therethrough, but which however retains cells and other large size compounds. The membrane used is preferably a ceramic membrane because they are highly resistant membranes. As a matter of fact, they can easily be sterilized through thermal or chemical means. They can easily be cleaned with chemical products or by pressure inversion (sending air or water).

These are properties that are not found in known organic membranes. Moreover, these membranes can be treated in an autoclave. The cutting threshold is precise. A ceramic membrane with a cutting threshold of one micron will not allow a molecule of a size higher than one micron to pass therethrough. Conversely, polymeric membranes could allow some molecules of one micron to pass therethrough. This notion is very important when it is question of extraction feasibility. Finally, ceramic membranes are certified to be safe in agri-food, cosmetic, nutraceutical fields and are of pharmaceutical grade.

The first filtration produces two portions: a concentrate mainly made of cells and an ultra-filtrate made of all the molecules not retained by the membrane. In the case of the production of secreted bio-molecules (exogenous) they are totally present in the ultra-filtrate. In the case of endogenous models, the bio-molecules of interest are found in the concentrate (in the cellular compartments). Their release is carried out by cellular lysis (sonication) and their extraction is carried out by membranous filtration that consists in separating the lysate into a concentrate (mainly composed of cellular debris and high molecular weight molecules) and an ultra-filtrate that contains the target molecule and the whole of the substances that pass through the membrane. In the case of the endogenous model, the first filtration (concentration of cells) allows on the one hand one to reduce the costs of lysis (sonication) and on the other hand to remove a large part of the organic and mineral substances that are present in the fermentation broth. In the two cases (exogenous and endogenous), the membranes used during this step of extraction (sequence) are not in a position to significantly separate the target bio-molecules and this in order to reduce the losses of target product.

In the two cases (exogenous and endogenous molecules), the concentration and purification (by diafiltration) that follow extraction may be carried out according to different membranous techniques that can ensure in part or totally, purification at the aimed level. The membrane that is appropriate for the operations of concentration and purification corresponds to the one that completely retains the target molecule (to minimize losses of target product) and that allows a maximum amount of impurities to pass therethrough. Diafiltration is an operation of purification that consists, once the desired concentration rate is reached, in pursuing the filtration by adding in the container, some concentrate of a diafiltration solution. The latter may either be demineralized water or a buffer solution. The addition of the diafiltration solution may be continuous (at a flow that is equivalent to that of the extracted ultra-filtrate) or discontinuous (addition in the concentrate and extraction by the ultra-filtrate of consecutive equal volumes). In diafiltration mode, the concentration of target molecule remains constant (the target molecule does not pass through the membrane and the volume of concentrate remains unchanged) and the compounds that are not retained by the membrane will keep on passing into the ultra-filtrate. The result is therefore an increase of the degree of purity. Purification should be optimized in order to use as little chemical products as possible. Among the membranous techniques used in this platform, micro-filtration, ultra-filtration and nano-filtration may be mentioned.

The finished product should be made of purified and concentrated bio-molecules.

The concentrate should contain micro-organisms and nutrients at high concentrations. pH, temperature, stirring and dissolved oxygen are also optimized.

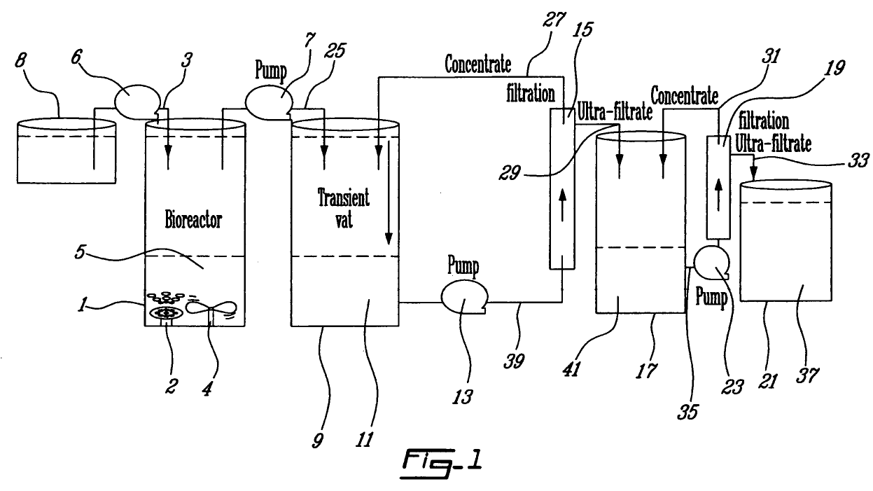

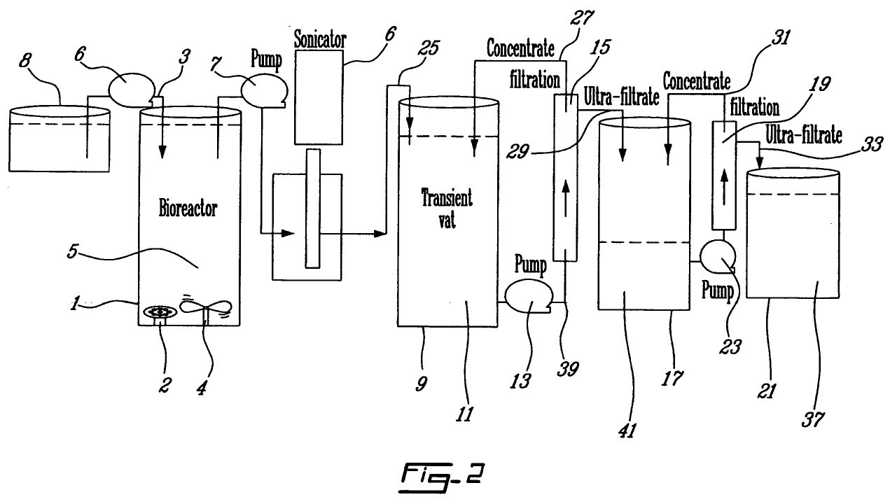

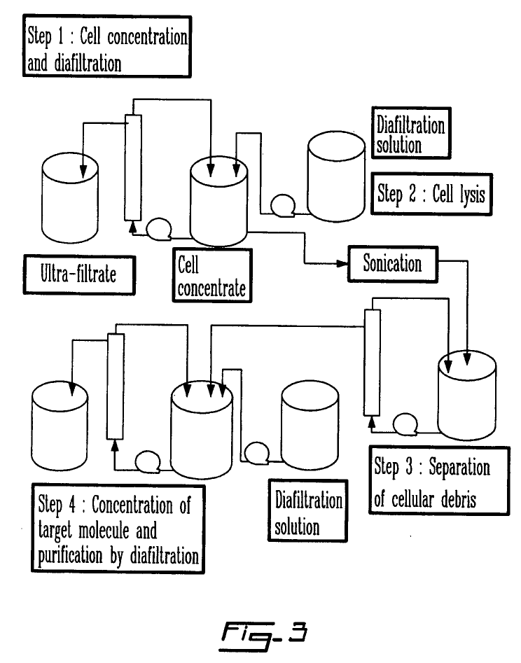

Referring now to FIGS. 1 and 2, a culture medium comprising a suspension of micro-organisms 5 is stored in a bioreactor 1. Nutrients are added to the suspension of micro-organisms 5 in the bioreactor by means of a duct 3. A ventilator 2 and a stirrer 4 respectively produce ventilation that is adapted to the micro-organism and the homogeneity of the suspension of micro-organisms 5.

A pump 5 provides for the transfer of a portion of the suspension of micro-organisms 5 in a transient vat 9 through a duct 25. A duct 29 located at the lower end of the transient vat 9 and a pump 13 provide for the continuous transfer of the suspension to be purified towards a primary filtration membrane 15. The concentrate of micro-organisms is sent towards the transient vat 9 through a duct 27 while the ultra-filtrate is sent towards a primary filtrate container 17 through a duct 29. The primary filtrate 41 is sent via a duct 35 and through a pump 23 towards a second filtration membrane 19. The concentrate is sent towards the primary filtrate container 17 via a duct 31 while the ultra-filtrate is sent towards the final solution container 21 through a duct 33.

Some of the characteristics of the system and of the method of production/separation of the present invention will be illustrated in the following example. However, it will be understood that this example is only presented as a complement and is not intended to limit or restrict the scope of the present invention.

### EXAMPLE 1

Production, Separation, Concentration and Purification of GFP

To validate this platform we have tested the endogenous and exogenous models of production of bio-molecules with the E-coli bacterium. The exogenous molecule model was tested by the production of β-lactamase. The production of GFP was the object of validation of the endogenous model. The molecular weights of GFP and of β-lactamase are respectively about 27 KD and 29 KD.

Continuous Fermentation

An optimization of continuous fermentation was carried out. Production parameters, dilution and volumes withdrawn were optimized, which has made it possible to limit the variability of the yields to less than 5%. The results of the yields obtained show that they are repetitive from one fermentation to the other and this applies for a number of fermentations that is higher than 30.

Separation, Concentration and Purification by Membranous Filtration

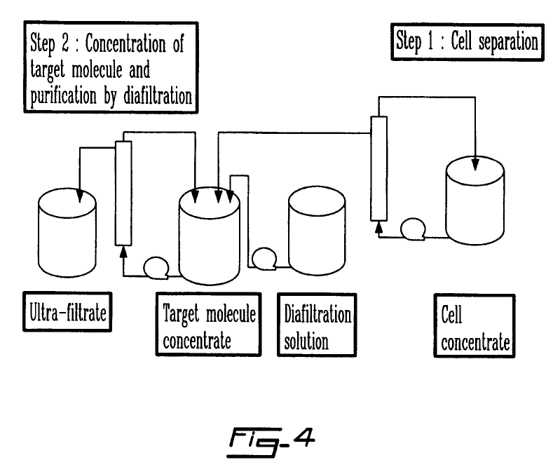

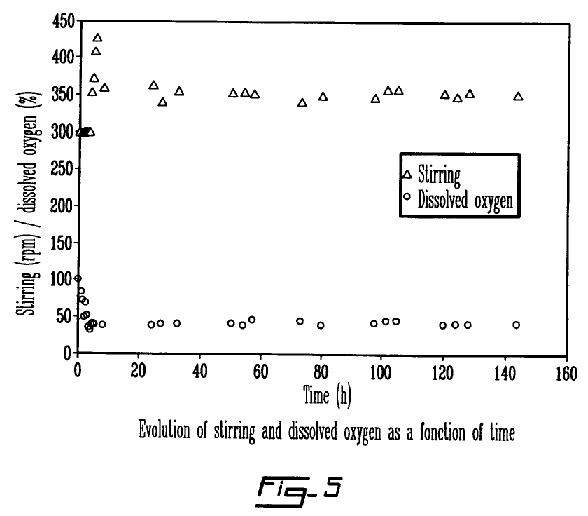

At the outlet of the bioreactor, the solution obtained is made of cells, various metabolites, nutrient substances that are not consumed, etc. GFP (target molecule) being endogenous, it is found inside the cells. The objective of membranous filtration aims at separating, concentrating and purifying the target molecule (GFP). As shown on FIG. 3, the process of separation, concentration and purification by membranous filtration is made of the following steps:
Step 1: Concentration and separation of the cells from the remainder of the medium.
Step 2: Lysis of the cells to release GFP from the cellular compartments.
Step 3: Separation of the cellular debris and macromolecules.
Step 4: Concentration of GFP and purification by diafiltration.

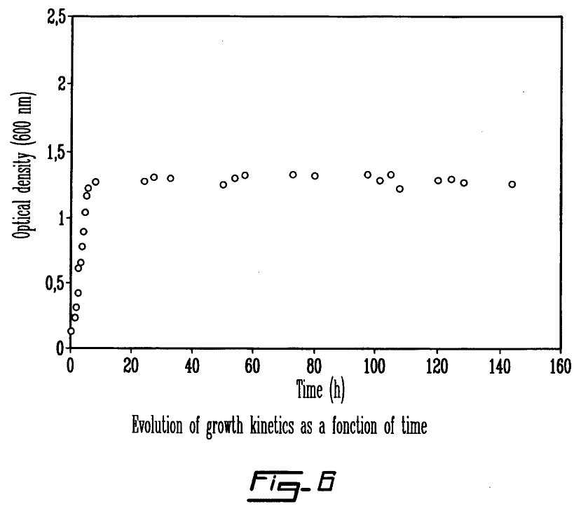

In the case of the exogenous molecule (β-lactamase), the membranous filtration sequences to achieve the separation, concentration and purification (diafiltration) are reduced to two steps (FIG. 4). The first one consists in the separation of the cells and the high molecular weight molecules. At the end of this step, the target molecule is found in the ultra-filtrate. The second step of filtration is a concentration of β-lactamase followed by a diafiltration to increase its purity.

Material and Methods

Membranous Filtration

The membranes used in the different filtration steps are ceramic membranes. This type of membranes is characterized by a strong resistance against chemical products and temperature and consequently, they answer the requirements of bio-processes (disinfection and sterilization). The membrane used in the first filtration step (separation and concentration of cells) is a micro-filtration membrane with a pore size of 0.2 μm. Separation of the cellular debris and of the macro-molecules (step 3) was carried out on a membrane with cutting threshold of 300 kD (MWCO). Concentration and purification (step 4) were carried out on an ultra-filtration membrane with a threshold cutting of 15 kD. This membrane allows one to efficiently concentrate (little loss) the target molecule and it allows a large proportion of dissolved matter of low molecular weight to pass therethrough. The membranes used originate from the company TAMI. The membranes used have an outer diameter of 2.5 cm (23 channels) and a length of 1.1 m. The filtering surface measures 0.35 m².

Operation Parameters

Concentration of the cells by membranous filtration was carried out at an average pressure of 16 psi (or 110 kPa), at a flow speed of about 20 l/min and a temperature of 7±1° C. Separation of the cellular debris was carried out at 6.2 psi (42 kPa), at a flow speed of 2 l/min and a temperature of 7±1° C. Concentration of GFP and diafiltration were carried out at an average pressure of 7.5 psi (52 kPa), a flow speed of 2 l/min and a temperature of 7±1° C. The average pressure is calculated from pressure measurements at the inlet and the outlet of the membranous module. All the filtration tests were carried at constant pressure. However, the filtration system used may be operated either at constant pressure (clogging produces a decrease of the flow of ultra-filtrate), or at constant flow of the ultra-filtrate (in this case, clogging is compensated by an increase of pressure).

Progress of a Filtration Operation

Each filtration operation on membrane included the following steps:
1. Determination of the permeability of the membrane with demineralized water. This determination is a characterization of the membrane in clean state.
2. Filtration of the solution to be treated. During filtration, the behavior of the membrane was determined by determinations of the production flow of the ultra-filtrate (evaluation of clogging of the membrane). Samples were taken to follow the evolution of the separation performances of the membrane.
3. Rinsing the membrane with demineralized water.
4. Determination of the permeability with demineralized water. This determination permits to evaluate clogging of the membrane.
5. Chemical washing of the membrane.
6. Determination of the permeability with demineralized water. The object of this determination is to evaluate the efficiency of the washing in reestablishing the initial state of the membrane (evaluating by determinations of permeability with demineralized water).
   Cell Lysis

Cell lysis was carried out with a sonication device operating in continuous. After an optimization study, the optimum conditions of sonication that were retained are: an intensity of 100% and a time of residence of 4 minutes. These conditions correspond to the best lysis ratio for the cells that are present in the concentrate without affecting the integrity of the target molecule (GFP).

Methods of Analyses

Growth of the cells through fermentation as well as separation and concentration of the cells by membranous filtration were determined from measurements of the optical density at a wavelength of 600 nm carried out by means of a Pharmacia Biotech Novaspec II spectrophotometer. The efficiency of this rapid method of analysis was validated by tests of culture on gelose. Determination of cells in the micro-filtration ultra-filtrate (step 1) was carried out by culture on gelose and this was achieved by reason of the low values of the optical density (DO at 600 nm) obtained. Performances of the membranous processes with respect to separation, concentration and purification of GFP were determined by fluorescence measurements. The apparatus used is a luminotox™ of the Lab-Bell™ company. Efficiency of diafiltration was followed through measurements of the total organic carbon (COT) by means of a Shimadzu TOC 5000A apparatus and of the total proteins (according to the Bradford method by using a standard curve with BSA). Analyses of different classes of proteins (Western-Blott in denaturant conditions) were carried out by means of electrophoreses.

Results

Stirring Optimization

The results obtained following optimization of this parameter suggest that the blades of Rushton type give better growth kinetics as compared to blades of the helical or helical+Rushton type. As compared to the Rushton blades, the helical type of blades have the disadvantage of generating a heterogeneous distribution of the air (oxygen) in the fermenter, the bubbles are of a large size which makes their distribution in the medium not very homogeneous and consequently, diffusion of oxygen has been found to be low in their case. On the other hand, the Rushton blades that have a more important shearing factor than the helical blades ensure a more homogeneous distribution of the air and consequently of the oxygen (the diffusion percentage of oxygen is higher with Rushton blades), the bubbles generated with this type of stirring are of a small size and are better distributed with respect to the other types that were tested. Diffusion of oxygen which follows the use of Rushton blades led to more rapid growth kinetics than those obtained with helical blades. Mixed blades give intermediate growth kinetics, between those obtained with the Rushton blades and the helical blades.

In this study, it results that the Rushton blades are the most appropriate for continuous fermentation with the recombinant E. coli strain used as bioreactor.

Determination of Stirring Speed

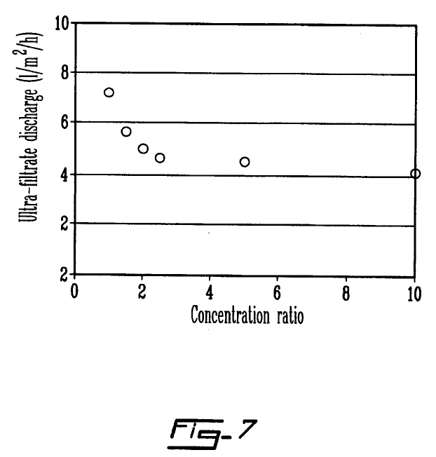

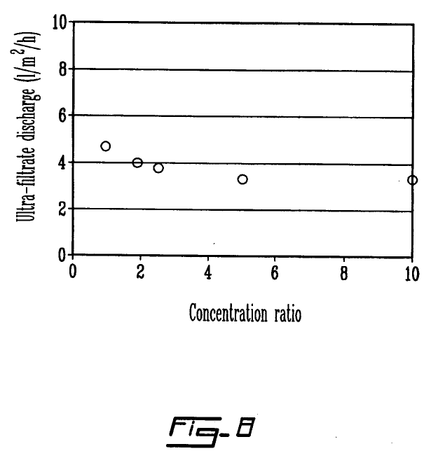

A range of stirring speeds that varies from 100 to 500 rpm was tested. The results (FIG. 5) obtained suggest that a stirring speed between 300 and 400 rpm generated the most rapid growth kinetics in its exponential phase. For example, a speed of 350 rpm gives growth kinetics 20% faster, during the exponential phase, as compared to a speed of 250 rpm. In both cases (250 and 350 rpm), there is equivalent formation of foam. The volume of anti-foam used during fermentations when the stirring speed was 350 rpm was 10% higher than the volume used with a stirring of 250 rpm.

Ventilation Optimization

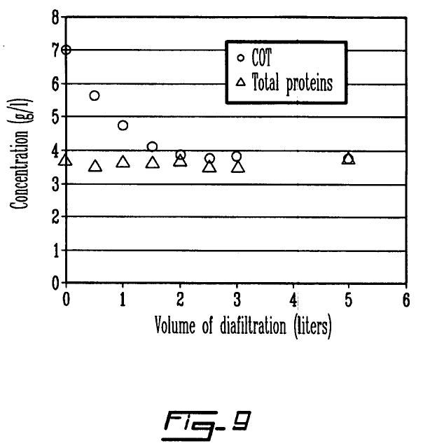

Ventilation of the fermenter (bioflo 110 of 14 liters with control terminal of the physico-chemical parameters) was ensured by a pump of the type Maxima R. The ventilation flows that were tested are 2 L/min, 3.5 L/min and 6 L/min. A flow of 6 L/min gave the best results represented by speed of growth and quantity of β-lactamase and GFP produced in a continuous mode. Production of β-lactamase was substantially equivalent in the case of a flow of 3.5 and of 6 L/min. On an economical point of view, the best correlation was obtained with a flow of 6 L/min when it is combined with a dilution volume of 37 ml/min in the case of a protein that is released in the (β-lactamase) or endogenous medium, i.e. GFP. In these two cases with this flow, during a plurality of fermentations (30), the DO obtained was stable and constant for more than six days in continuous mode (FIG. 6).

Cell Concentration (Step 1)

The first filtration step on a membrane is a micro-filtration that consists in separating the cells from the remainder of the solution. This filtration therefore produces a concentrate (mainly made of cells) and an ultra-filtrate, which is made of the whole of the substances not retained by the micro-filtration membrane (buffer, nutrient substances, etc.). The target molecule being endogenous, at this stage of the operation, it is therefore inside the cells. The membrane used in this filtration step is a ceramic membrane whose pore size is 0.2 μm. Once the desired rate of concentration is reached, a diafiltration with a PBS buffer (Na₂HPO₄, 8 mM; NaH₂PO₄, 2 mM and NaCl, 0.14 mM) is carried out to reduce the concentration of the organic and mineral compounds in the cell concentrate. The diafiltration volume was fixed at twice the volume of the concentrate (i.e. 30 liters). Table 1 summarizes the results of this operation. The results (table 1) show that cell concentration is complete, it corresponds to the rate of concentration determined from the volumes (initial volume/volume of concentrate). The rate of concentration of DO at 600 nm is lower which indicates a removal of part of the dissolved material (about 20% of DO at 600 nm). Removal of COT is 87%. DO at 600 nm still decreases under the effect of diafiltration, thus improving the rate of removal, of the dissolved material that is present in the cell concentrate (30% removal of initial DO).

With respect to the clogging of the membrane, filtration of the culture broth leads to a rapid decrease of the flow of ultra-filtrate by more than 90% with respect to the flow measured with demineralized water (including the effect of viscosity since demineralized water permeability was carried out at 25° C.). This rapid loss is followed by a small gradual loss of the flow of ultra-filtrate under the effect of concentration. The flow of ultra-filtrate varied between 24 l/m²/h at the start of the concentration and about 17 l/m²/h at the end of the concentration. Clogging is of reversible nature since water rinsing managed to recover about 50% of the permeability of the membrane and a wash with a solution of sodium hypochlorite has entirely restored the initial permeability of the membrane.

TABLE 1
Results of cell concentration by membranous filtration

| | | Final | | |
|---|---|---|---|---|
| | Initial | ultra- | Final | Concentra- |
| | solution | filtrate | concentrate | tion ratio |
| DO at 600 nm | 1.335 | | 10.75 (9.25) | 8 |
| (cm⁻¹) | | | | (6.9) |
| Hymacymetry | 10⁹ | | 10¹⁰ | 10 |
| (c/ml) | | | | |
| Humid biomass | | | 30.8 | |
| (g/l) | | | | |
| Total proteins | <100 | | | |
| (mg/l) | | | | |
| Organic carbone | 2800 | | 3654 | |
| (mg/l) | | | | |
| Volume (liters) | 150 | 135 | 15 | 10 |

( ) DO at 600 nm mesured after diafiltration.

Cell Lysis by Sonication (Step 2)

This step consists in a cell lysis to release GFP (endogenous molecule) from the cellular compartments. The process that is chosen to carry out cell lysis is sonication. The sonication parameters (intensity and duration of sonication) were optimized so as to obtain the best lysis yields while preserving the integrity of the target molecule (GFP). Sonication was carried out on the cell concentrate produced in the first filtration (step 1) to which a re-suspension buffer (buffer P1: Tris-HCl, 550 mM, pH 8; EDTA 100 mM; sodium azide 0.2% (P/V) is added. Volume of the buffer represents 10% of the total volume of solution. The results of performances of sonication are summarized in table 2. The conditions of sonication applied have allowed the lysis of 83% of the cells. The results show a release of an important quantity of proteins (measurement of total proteins) and an increase of the organic carbon (part of COT is brought in by the buffer) in the lysate. It should be noted that the measurements of COT and total proteins were carried out after centrifugation of the samples at 14000 g during 20 minutes.

TABLE 2
Results of cell lysis by sonication

| | Concentrate | | |
|---|---|---|---|
| | before | | |
| Cell lysis | sonication | Lysate | Efficiency |
| Cell count (c/ml) | 10¹⁰ | 1.7 × 10⁹ | 83% |
| DO at 600 nm (cm⁻¹) | 9.25 | 8.95 | |
| Fluorescence with debris | | 0.7 | |
| Fluorescence without | | 0.08 | |
| debris⁽*⁾ | | | |
| Total proteins⁽*⁾(mg/L) | 335 | 537 | |
| Organic carbon (mg/l) | 3650 | 5400 | |
| Volume (liters) | 15 | 15 | |

⁽*⁾the total proteins and fluorescence without debris and organic carbon were measured after a centrifugation of the samples at 14000 g during 20 min.

Separation of Cell Debris (Step 3)

After release of the target molecule from the cellular compartments (step 2), the solution is given a second membrane filtration whose objective is to separate cellular debris and high molecular weight molecules from the remainder of the solution. This filtration therefore produces a concentrate, which is essentially made of cellular debris, and an ultra-filtrate containing the target molecule and the remainder of the low molecular weight compounds. The performances of this step of the process are summarized in table 3. FIG. 3 shows the evolution of the ultra-filtrate as a function of the concentration ratio (initial volume/volume of concentrate) during filtration.

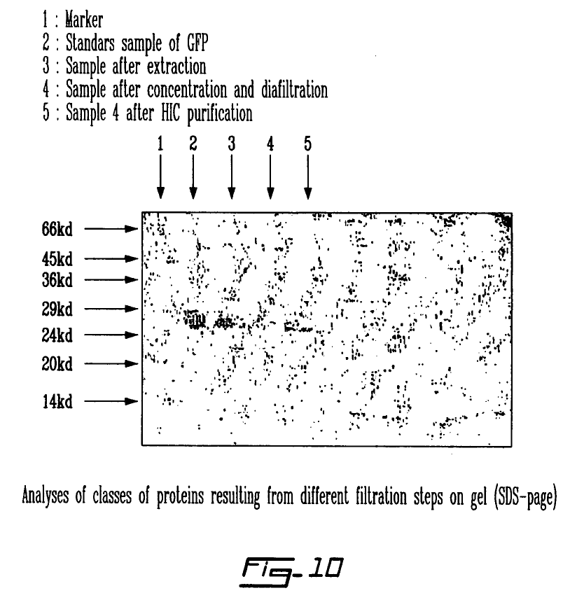

The results of the table show that the membrane with a 300 kD of cutting threshold allows a total removal of the cellular debris (measurement of DO at 600 nm) and a reduction of the quantity of total proteins and of COT by 41% and 16% respectively and this as compared to centrifugation at 14000 g during 20 minutes. The loss of GFP in the concentrate (estimated from the total amount of calculated material from measurements of fluorescence) is about 15%. However, a more important volume of diafiltration would allow a reduction of the loss ratio of GFP in the concentrate. With respect to membrane clogging (FIG. 7), two observations may be made: the flow of ultra-filtrate is characterized by a rapid loss of more than 90% (including the effect of viscosity due to the fact that measurement of the permeability with demineralized water was carried out at 25° C.) after a few minutes of filtration (as compared to the one measured with demineralized water). This is followed by a gradual decrease with an increase of the concentration ratio that is stabilized at about 4 l/m²/h. Permeability of the membrane was restored to its initial level by means of a chemical wash (sodium hypochlorite solution).

TABLE 3
Separation of cellular debris by membranous filtration

| | | Concen- | Ultra- | Removal |
|---|---|---|---|---|
| Separation of debris | Lysate | trate | filtrate | ratio |
| Cell count c/ml) | 1.7 × 10⁹ | | | |
| DO at 600 nm | 8.95 | 137 | 0.001 | 100% |
| (cm⁻¹) | | | | |
| Fluorescence | 0.7 | 8.86 | 0.067 | |
| with debris | | | | |
| Fluorescence | 0.08⁽*⁾ | 0.170 | 0.067 | 15%⁽**⁾ |
| without debris | | | | |
| Total proteins | 537⁽*⁾ | 2286⁽*⁾ | 308 | 41% |
| (mg/L) | | | | |
| Organic carbon | 5400⁽*⁾ | 5740⁽*⁾ | 4424 | 16% |
| (mg/l) | | | | |
| Volume (liters) | 10 | 0.7 | 9.3 | |
| | | | (10.3) | |

⁽*⁾total proteins and fluorescence without debris and organic carbon were measured after centrifugation of the samples at 14000 g during 20 min.
⁽**⁾percentage of loss of fluorescence.
( ) volume of ultra-filtrate after diafiltration with 1 liter of demineralized water.

Concentration of Target Molecule (GFP) and Diafiltration (Step 4)

This filtration step aims on the one hand at concentrating GFP and on the other hand at increasing purity by diafiltration. The objective of the concentration operation is to reduce the volume of the solution with a minimum of loss of the target molecule (GFP). The choice of the most appropriate membrane for carrying out this operation corresponds to the one which permits to efficiently concentrate the target molecule (minimize losses) and remove the maximum amount of impurity. The results obtained are summarized in the table. To be noted is the efficiency of the membrane used in concentrating GFP (loss ratio of about 70%). Moreover, the membrane has allowed removal of 87% of the COT present in the initial solution. With respect to clogging of the membrane during the concentration operation, the evolution of the flow of ultra-filtrate with respect to the concentration ratio (FIG. 8) is characterized by a rapid decrease of about 84% (as compared to the flow of the ultra-filtrate measured with demineralized water at the same pressure and at a temperature of 25° C.) followed by a small gradual decrease under the effect of concentration. The flow of ultra-filtrate is stabilized at about 3 l/m²/h (pressure of 7.5 psi and a temperature of 7±1° C.).

Purification by diafiltration is carried out on the same filtration system as step 4 (FIG. 1). Once the desired concentration ratio is reached (step 4), we start up the filtration in diafiltration mode. The latter consists in the addition in the concentrate container of a buffer solution (TE: Tris-HCl, 10 mM, pH8; EDTA, 1 mM; sodium azide 0.02%). The objective of the diafiltration is to increase the degree of purity of the target molecule (GFP) in the concentrate. Indeed, in diafiltration mode, the concentration of GFP remains constant (the target molecule does not pass through the membrane) and the compounds which are not caught by the membrane will continue to pass in the ultra-filtrate. The result is therefore an increase of the degree of purity. The function of the buffer is to preserve the integrity and the stability of GFP. The results of table 5 show the characteristics of the concentrate before and after diafiltration. FIG. 9 shows the COT evolution and that of the total proteins in the concentrate with respect to the volume of diafiltration. An important decrease of the COT until reaching a diafiltration volume of 1.5 liters may be observed. Diafiltration is not efficient with respect to the removal of the total proteins (FIG. 9). Indeed, a gel analysis has shown that most of the total proteins that pass through the 300 kD membrane are between 15 and 70 kD (FIG. 10) and consequently, their concentration through the 15 kD membrane is nearly total. With respect to clogging, diafiltration produces no supplementary loss of membrane permeability. Indeed, the flow of ultra-filtrate during diafiltration remains stable and similar to that registered at the end of the concentration step (i.e. about 3 l/m²/h). As for the 0.2 μm and 300 kD membranes, the initial permeability of the membrane was restored by chemical washing (sodium hypochlorite solution).

TABLE 4
Concentration of target molecule (GFP) by membranous filtration

| | Lysate | | | |
|---|---|---|---|---|
| | filtered | | | |
| Concentration of | on | Concen- | Ultra- | Removal |
| GFP | 300 kD | trate | filtrate | ratio |
| DO at 600 nm (cm⁻¹) | 0.001 | | | |
| Fluorescence | 0.067 | 0.448 | 0.005 | 7% |
| Total proteins (mg/L) | 308 | 3816 | <100 | 0% |
| Organic carbon (mg/1) | 4424 | 7000 | 4200 | 87% |
| Volume (liters) | 10 | 0.8 | 9.2 | |

TABLE 5
Purification by diafiltration

| | | Concentrate | |
|---|---|---|---|
| | | at the end of | Removal |
| Diafiltration | Concentrate | diafiltration | ratio |
| DO at 600 nm (cm⁻¹) | 0.277 | 0.015 | |
| Fluorescence | 0.448 | 0.491 | |
| Total proteins (mg/L) | 3816 | 3800 | 0% |
| Organic carbone | 7000 | 3670 | 48% |
| (mg/l) | | | |
| Volume (liters) | 0.8 | 0.8 | |

Micro-filtration on a membrane of 0.2 μm porosity makes it possible to concentrate all the cells that are present in the fermentation broth and to considerably reduce the volume of the solution. It also contributes to the removal of an important part of the dissolved matter (20% of the DO at 600 nm and 87% of the COT). Moreover, the removal ratio of the dissolved matter may be increased by diafiltration.

With respect to the release of GFP from the cellular compartments, sonication made it possible to reach a lysis ratio of 83%. It should be noted that in addition to the release of GFP, there is release of an important quantity of proteins.

Separation of the cellular debris on a membrane with a cutting threshold of 300 kD leads to a total removal of the cellular debris (total elimination of DO at 600 nm). Moreover, it holds back 41% of the total proteins and 16% of the COT and this as compared to a centrifugation at 14000 g during 20 minutes.

1. Process for the continuous production and separation of target polypeptides with a micro-organism, comprising the steps of:
a) causing the growth of a micro-organism producing at least one target polypeptide in a bioreactor that is autonomously supplied with a culture medium for a predetermined period of time;
b) causing a portion of the culture medium to be autonomously transferred in a first container, in a manner that this container promotes the accumulation of volumes extracted from the bioreactor in compensation for continuously added volumes of fresh medium, the container being refrigerated at 4° C. to allow for the accumulation and the residence of a mixture of cells and nutrient broth before undergoing sonication, a second refrigerated container of same capacity and kind as the first container serving to preserve the mixture at 4° C. after sonication;
c) autonomously inducing separation of the target polypeptides from portion of culture medium through at least one passage in at least one first membrane, selected (nature and cutting threshold) according to filtration objectives, the characteristics of the solution to be filtered and the operation criteria, preferably ceramic filtration or ultra-filtration membranes;
d) separating the target polypeptides from the microbial molecules by passage through at least one second membrane; and
e) collecting the target polypeptides.

2. Process according to claim 1, characterized in that said bioreactor is a bioreactor with external membrane.

3. Process according to claim 1, characterized in that said target polypeptides are proteins.

4. Process according to claim 3, characterized in that said proteins are enzymes, transport proteins, anti-oxidizing proteins or food proteins.

5. Process according to claim 1, characterized in that said micro-organism is a bacteria, yeast, mildew, a virus, a protozoan, or a fungus.

6. Process according to claim 1, characterized in that said target polypeptides are recombinant polypeptides.

7. Process according to claim 1, characterized in that said target polypeptides are polypeptides (recombinant proteins) which are endogenous towards said microorganism.

8. Process according to claim 1, characterized in that said micro-organism is replaced by yeast, a plant cell, an animal cell, or an insect cell.

9. Process according to claim 1, characterized in that said growth corresponds to a point at the end of the logarithmic growth phase, with an optical density (DO) of 1.8, at a concentration of 0.2×10¹⁰ cells per milliliter, this cellular concentration being obtained between 7 and 8 hours after the start of the bio-fermentation and 2 hours after the start up of the pumps provided for the transfers between the bioreactor and the first and second containers, the operation of the pumps adapted for adding fresh medium being carried out at a flow of 37 ml/min, for the type of fermenter used, and optimized for purging at an equivalent flow, and the average time for starting up the pumps was determined at 5 h 30 after the start of the fermentation, the DO being equal to 1.5, the DO and the cellular concentration remaining stable and constant (variability <10% calculated on a basis of 30 fermentation) during the entire step of production in the fermenter.

10. Process according to claim 1, characterized in that said portion of culture medium of step b) contains the target polypeptide alone, or the micro-organism containing the target polypeptide in endogenous manner.

11. Process according to claim 1, characterized in that said culture medium contains the nutrient substrates that are essential for the growth of said micro-organism.

12. A device for the continuous production of at least one target polypeptide comprising:
a bioreactor;
a source of culture medium and of nutrient substrates, and means allowing their introduction in the bioreactor;
means for ventilating the bioreactor;
means for stirring the culture medium in the bioreactor;
a first container adapted for providing a sterile preservation of the culture medium and means to allow its autonomous and continuous transfer in said bioreactor;
at least one first filtration means (micro-filtration) and means for separating the cells (biomass) that are present in the concentrate from the partially consumed culture medium (ultra-filtrate) by a filtration means (micro-filtration) from the bio-fermenter; means operative so that after membranous separation, the concentrate remains in the first container and the ultra-filtrate is sent towards a second container, this second container being arranged to constitute a suspension of filtered target polypeptide;
means for introducing the suspension of filtered target polypeptide in the second container after a first filtration, the second container permitting collection and residence of the ultra-filtrate under sterile conditions at a temperature of 4° C., this ultra-filtrate constituting the portion that contains the target protein when it is release in the culture medium or in the cellular compartment, the latter being released after lysis by sonication, the separation being carried out after the sonication step, the target polypeptide being found in the ultra-filtrate after a separation by micro-filtration;
at least one second filtration means (ultra-filtration ceramic membrane) and means for introducing the suspension of polypeptide having undergone a first filtration in the second filtration means from the second container; and
means for collecting the target polypeptide that has been subjected to a first and a second filtration.

13. Device according to claim 12, characterized in that said bioreactor is a bioreactor with external membrane.

14. Device according to claim 12, characterized in that the first filtration means is a membrane.

15. Device according to claim 14, characterized in that said membrane is a ceramic membrane.

16. Device according to claim 12, characterized in that said second filtration means is a membrane.

17. Device according to claim 16, characterized in that said membrane is a micro-filtration, ultra-filtration, nano-filtration, reverse osmosis or dialysis membrane.
